(19) United States
(12) Patent Application Publication
(10) Pub. No.: US 2012/0088240 A1
(43) Pub. Date: Apr. 12, 2012

(54) FUNCTIONALIZED POLYMER BIOSENSOR

(76) Inventors: Christina O. BAKER, Los Angeles, CA (US); Chris BEHRENBRUCH, Brunswick (AU); Lola RAHIB, Los Angeles, CA (US)

(21) Appl. No.: 13/279,509

(22) Filed: Oct. 24, 2011

Related U.S. Application Data
(63) Continuation of application No. PCT/US2010/032329, filed on Apr. 24, 2010.
(60) Provisional application No. 61/214,523, filed on Apr. 24, 2009.

(51) Int. Cl. C12Q 1/68; C08G 73/02; B82Y 15/00

(52) U.S. Cl. 435/6.11; 525/54.2; 977/773; 977/755; 977/762; 977/902

(57) ABSTRACT

One aspect of the present disclosure relates to a novel sensor mechanism based on the aggregation of nanoparticles for target molecule detection and quantification. The nanoparticles that can be used include non-conducting polymers and conducting polymers such as polyaniline, polypyrrole and polythiophene derived nanofibers. Embodiments can include covalently functionalized nanoparticles with probes for target molecules, a biosensor where functionalized nanoparticles bind to one another upon presence of target to generate a visible conjugate induced aggregation, a biosensor wherein nanoparticles bind spontaneously in the presence of target molecules such as biological molecules, cells and biological markers.

### PRIORITY CLAIM

The present application is a continuation of International Application No. PCT/US2010/032329, filed Apr. 24, 2010, and designating the U.S., which claims priority to U.S. Provisional Application 61/214,523, filed Apr. 24, 2009, both applications of which are incorporated herein by reference in its entirety.

### STATEMENT REGARDING FEDERALLY SPONSORED RESEARCH OR DEVELOPMENT

The invention was made with Government support under Grant No. IIP-0930767 awarded by the National Science Foundation. The Government has certain rights in the invention.

### FIELD

This invention relates to the field of nanotechnology, biosensors and diagnostic devices, and detecting methods thereof.

### BACKGROUND

Conventional biomolecule detection (i.e. nucleic acid hybridization techniques, polymerase chain reaction (PCR), quantitative PCR, southern blotting, northern blotting, western blotting, cell capture, etc.) requires complicated processes or labeled substances that may only be quantified by special instrumentation. Tests for bacterial infections are typically done clinically through microscopy and culture of specimen, followed by bacterial analysis to determine the antibiotic susceptibility of the pathogenic bacteria and the details of the infection. Because this process relies on bacterial culture, result times are often lengthy (2-3 days) and costly (>$100/test).

More rapid tests are commercially available over-the-counter, but the poor accuracy of these devices causes difficulty for using them in diagnostics. For example, urinary tract infection (UTI) tests in the form of a urine dipstick can detect leukocytes and/or nitrites, which are secondary indicators of the presence of uropathogens. These kits are plagued by frequent false-negatives and false-positives, significantly lessening their impact in accurately indicating an infection.

The more expensive and time-consuming bacterial culture step has a few competing technologies in development. Electrochemical detection and Polymerase Chain Reaction (PCR) are the most common and utilized for accurate analysis of pathogens or biomarkers.

The goal of PCR is to amplify copies of a specific DNA target. Specific gene detection or absence can lead to identification of the infection-causing bacteria. Despite efforts to make PCR more robust and less expensive (Goel, 2006) it is still a costly technique due to its consumables and instrumentation.

Electrochemical detection is another means for detecting biological molecules and offers the benefit of multiple analyte detection (Labgold, Jokhadze, Jen, Shen, Kozlowski, Ammini, Suhy, Norris and Lobban, 2007). For example, electrochemical detection for bacterial analysis makes use of sequence specific probes complimentary to 16S rRNA targets (Haake, Churchill, Liao, Suchard, Li and Mastali, 2008,Liao, Haake, Churchill and Suchard, 2006,Liao, Mastali, Gau, Suchard, Moller, Bruckner, Babbitt, Li, Gornbein, Landaw, McCabe, Churchill and Haake, 2006,Liao, Mastali, Li, Gau, Suchard, Babbitt, Gornbein, Landaw, McCabe, Churchill and Haake, 2007,Mastali, Babbitt, Li, Landaw, Gau, Churchill and Haake, 2008, Sun, Liao, Zhang, Gau, Mastali, Babbitt, Grundfest, Churchill, McCabe and Haake, 2005) for multiplexed sensing of bacteria in UTI samples. This method provides rapid analysis of urine samples through DNA/rRNA hybridization reactions. The technology utilizes a DNA-functionalized electrode surface, target 16S rRNA from urinary bacteria, and a horseradish peroxidase (HRP) functionalized DNA probe to generate a redox chemistry-based electrical signal when the probe-target-detector probe hybridization occurs (Gao, Xie and Yu, 2005). Although HRP is a commonly used signaling molecule, others have been explored such as photoactivated organic molecules (Gao, 2007). Additionally, systems have found success with probes and linkers from peptides nucleic acid (PNA) (Achim, Shi and Yeh, 2009), locked nucleic acid (LNA), and other nucleic acid derivatives. One of the drawbacks of electrochemical detection for UTI diagnosis is the cost; the method typically requires an expensive multiplexing potentiostat and self-assembled monolayer (SAM) functionalized gold electrode consumables (Haake, et al., 2008).

Polyaniline and conducting polymers have been used by some researchers to detect DNA and other biological molecules. This is typically done with conventional forms of polyaniline that are synthesized in the form of micron scale particles that are ground, dissolved in a strong solvent, filtered and then combined with a redox active molecule to generate an electrical signal in the presence of the target(Musho, Noell and Tse, 1992). Polyaniline nanofibers synthesized electrochemically have also been used in DNA sensing through the use of covalent attachment of carboxylated probes to the amine terminus of electrochemically or template grown polyaniline nanofibers using 1-ethyl-3-(3-dimethylaminopropy-1)-carbodiimide (EDC) and N-hydroxysuccinimide (NHS) or other coupling agents (Chang, Yuan, Shi and Guan, 2007,Yang, Zhou, Zhang, Zhang, Jiao and Li, 2009,Zhu, Chang, He and Fang, 2006). This method limits the degree of functionalization possible to one per polymer chain, and only at the chain terminus. Other conducting polymers, such as polypyrrole, have been functionalized with DNA using carboxylic acid side groups on the monomer, with similar coupling chemistry. These methods have not been expanded for use with polyaniline or conducting polymer nanofibers synthesized with scalable methods (Livache, Roget, Dejean, Barthet, Bidan and Teoule, 1995,Livache, Roget, Dejean, Barthet, Bidan and Teoule, 1994,Wang, Jiang, Fortes and Mukherjee, 1999).

There is a high demand for inexpensive, fast, and accurate detection of biomolecules leading to the detection of a wide-variety of diseases and infections. Optical methods of detection to determine the presence, absence, or amount of target biological molecules provide a potential for the much-needed cost effective detection of infectious diseases, genetic disorders, newborn screening, prenatal screening, sexual transmitted diseases (STDs), cancer, and pathogens in the food/water/waste stream.

### SUMMARY

One aspect of the invention relates to a biosensor comprising a nanoparticle-based detection unit functionalized with a nanoparticle-based capture unit, wherein: the nanoparticle-based detection unit comprises a nanoparticle; the functionalization is through covalent or non-covalent interaction; the nanoparticle-based capture unit comprises one or more capture probes, which can be the same or different types of capture probes, and which can conjugate with the same or different target molecules; the conjugation of target molecules with the capture probes of the nanoparticle-based capture unit result in a change of the nanoparticle-based detection unit, which provides a nanoparticle-based detection signal; and the change of the nanoparticle-based detection unit is selected from the group consisting of a morphological change, a color change, an optical change, an electrochemical change, and any combination thereof.

The target molecule can be selected from the group consisting of antibodies, peptides, enzymes, proteins, small molecules, cells, biological markers, DNA, PNA, LNA, RNA, derivatives thereof, an indicator of an infectious disease, an indicator of a non-infectious disease, an indicator of a genetic disorder, an indicator of a newborn screening, an indicator of a prenatal screening, an indicator of a sexual transmitted disease, an indicator of a cancer, an indicator of the presence of pathogens, DNA, RNA, proteins, peptides, small molecules and any combination thereof, and cooligomers and any combinations thereof.

The change of the nanoparticle-based detection unit provides a detection signal which can be determined by naked eye or by equipment (e.g. optical read-out equipment, microscope, UV-vis spectrophotometry, dynamic light scattering, zeta potentiometers, viscometers, cyclic voltammogram, current-voltage read-out equipment, impedance read-out equipment, and potentiostat).

As used herein, the term “morphological change” means a change in morphology of the nanoparticle-based detection unit. For example, in the absence of target-molecules, the biosensors and the nanoparticle-based detection units thereof are dispersed in a solution to form a semi-stable uniform suspension. In the presence of the target-molecules, the nanoparticle-based detection units aggregate together through conjugations of the capture probes and the target molecules. The morphology of the nanoparticle-based detection unit has changed from uniformly dispersed in the solution to biosensor-sample conjugates. The morphological change can be detected by equipment or by naked eyes if the change is significant (e.g. precipitation of the biosensor-sample conjugates).

In certain embodiments, optical change of the nanoparticle-based detection unit also occurs with the formation of the biosensor-sample conjugates. For example, the scattering profiles of biosensor-sample conjugates are different comparing to that of the uniform dispersion. Another example of optical change is color change caused by the conjugation of target molecules and the capture probes.

The formation of biosensor-sample conjugates can also cause electrochemical change. For example, in certain embodiments, electrochemical change caused by the formation of biosensor-sample conjugates can be detected directly when the biosensors comprise conducting polymer nanofibers functionalized (impregnated or covalently incorporated) with capture probes specific to the target molecules and detection probes that are redox active (e.g. horseradish peroxidase (HRP)). The redox active detection probe can be directly read out.

Another aspect of the invention relates to a biosensor system comprising the biosensor and a signal enhancer which can conjugate with the biosensor to enhance the detection signal.

Another aspect of the invention relates to a biosensor system comprising the biosensor and a biosensor substrate-based capture unit, wherein the substrate-based capture unit comprises: a substrate functionalized with one or more types of capture probes; the capture probes can conjugate with the target molecules of the biosensors; and the capture probes are deposited in a single spot on the substrate, or in an array on the substrate.

Another aspect of the invention relates to a method of detecting a target molecule from a sample comprising: providing a biosensor or a biosensor system comprising a biosensor according to the present disclosure; contacting the biosensor or a biosensor system with the sample to provide a biosensor-sample conjugate or a biosensor system-sample conjugate; and detecting a detection signal of the biosensor-sample conjugate or the biosensor system-sample conjugate to determine the presence or concentration of the target molecule in the sample.

### BRIEF DESCRIPTION OF THE DRAWINGS AND TABLES

FIG. 1. Preparation of a biosensor using carboxylic acid functionalized polyaniline.

FIG. 2. DNA-polyaniline nanofibers biosensors suspended in solution aggregate after binding to the target biological molecule (rRNA) from bacteria and no longer remain suspended in solution.

FIG. 3. Detection of target molecules using a biosensor system: (a) a substrate-based capture unit, a type of capture probes for a different target molecule was deposited in each spot on the array of the substrate; (b) biosensors dispersed in a solution (left) and a sample containing target molecules was provided (right); (c) mixing biosensors with the sample to form biosensor-sample conjugates, which were dispersed in the solution; (d) contacting biosensor-sample conjugates with the substrate to form substrate-biosensor-sample conjugates, which altered the optical (e.g. color) and/or electrochemical properties of the substrate and provided an electrical and/or optical read out.

FIG. 4. Illustration of substrate-biosensor-sample conjugates and signal amplification (I): conjugation of a first type of biosensors (Biosensor 1) comprising a first type of capture probes (Capture probe 1), a substrate comprising a second type of capture probes of substrate-based capture unit (Capture probe 2) and target molecules together in a sandwich arrangement to provide a read out signal (left); amplification of the signal by addition of a second type of biosensors (Biosensor 2) functionalized with a third type of capture probes (Capture probe 3) to enhance the level of conjugation among probes, nanoparticles, substrate and target molecules (right).

FIG. 5. Illustration of substrate-biosensor-sample conjugates and signal amplification (II): conjugation of a first type of biosensors (Biosensor 1) comprising a first type of capture probes (Capture probe 1) and a first type of detection probe (Detection probe 1), a substrate comprising a second type of capture probes of substrate-based capture unit (Capture probe 2) and target molecules together in a sandwich arrangement to provide a read out signal, (left); amplification of the signal by addition of a second type of biosensors (Biosensor 2) functionalized with a second type of detection probes (Detection probe 2) that conjugates with the first type of detection probes to enhance the level of aggregation of biosensor nanoparticles (right).

FIG. 6. (A) Relationship between sheet resistance and degree of COOH/PANI functionalization. (B) Relative absorbance (COOH/PANI) in all samples incorporated with COOH.*P value <0.005, Student's t-test. (C) Cyclic Voltammetry showing changes of oxidation state of functionalized-PANI compared to naked PANI.

FIG. 7. An electrochemical array for deposition of probe functionalized conducting polymers. The central electrode is the working electrode, and one of the outer electrodes is the counter electrode and the others a reference electrode.

FIG. 8. Amplification of the signals provided in DNA capture probe detection systems using HRP and hydrogen peroxide (H₂O₂).

FIG. 9. Detection using conducting polymer nanofibers through direct interaction between a conducting polymer and the desired capture probe during polymer synthesis.

FIG. 10. Conjugation of PANI-probe1 and PANI-probe2 via target RNA samples from E. coli showed precipitation in the reaction.

FIG. 11. Conjugation of PANI-probe1 and PANI-probe2 via target RNA of lysed cells from E. coli showed precipitation in the reaction.

FIG. 12. Conjugation of PANI-probe1 and PANI-probe2 via target RNA of lysed urine samples showed precipitation in the reaction.

FIG. 13. Aggregation of polyaniline nanofiber biosensors with the presence of Leukocytes.

FIG. 14. Microscopic (10×) image of polyaniline biosensors conjugated with E. coli RNA and further conjugated with a glass substrate functionalized with probes of the same target.

### DETAILED DESCRIPTION

### I) Biosensor and Preparation Thereof

### A) Structure of Biosensor

One aspect of the invention relates to a biosensor comprising a nanoparticle-based detection unit functionalized with a nanoparticle-based capture unit, wherein:
the nanoparticle-based detection unit comprises a nanoparticle;
the functionalization is through covalent or non-covalent interaction;
the nanoparticle-based capture unit comprises one or more capture probes, which can be the same or different types of capture probes, and which can conjugate with the same or different target molecules;
the conjugation of target molecules with the capture probes of the nanoparticle-based capture unit result in a change of the nanoparticle-based detection unit, which provides a nanoparticle-based detection signal; and
the change of the nanoparticle-based detection unit is selected from the group consisting of a morphological change, a color change, an optical change, an electrochemical change, and any combination thereof.

As used herein, the term “functionalization” means attachment of a first subject to a second subject via covalent and/or non-covalent interaction(s).

As used herein, the term “non-covalent interactions” are interactions that do not involve the sharing of pairs of electrons, e.g. hydrogen bonds, ionic bonds, van der Waals forces, and hydrophobic interactions.

As used herein, the term “subject” means a molecule, a cell, a sample, a material, a substrate, a target molecule, a capture probe, a detection probe, a derivative, aggregation or combination thereof.

Examples of a sample include, without limitation, water, food, blood, urine, cerebrospinal fluid, mucous, biopsies, biological specimens, bodily fluids and lysis thereof to release target molecules.

Examples of a substrate include, without limitation, a nanoparticle, a dipstick array, a porous substrate, a transparent substrate, a substrate coated with a transparent conductor substrate, a transparent conductor electrode substrate of an electrochemical monitoring equipment, a glass substrate, a silicon substrate, a mica substrate, a metal substrate, an indium tin oxide (ITO) substrate, a cloth substrate, a paper substrate, a cotton substrate, a wood substrate, a cellulose substrate, polymeric film, a nitrocellulose substrate, a polyvinylidene fluoride substrate, a polystyrene sulfonic acid substrate, a poly-lysine substrate, a conductive substrate, a carbon nanotubes substrate, a graphene substrate, a silver nanowire substrate, a transparent conductor electrode substrate of potentiostat, and any combination thereof. In certain embodiments, a substrate can be coated with another substrate, such as ITO coated glass, mica, silica or plastic substrate.

Examples of target molecules include, without limitation, antibodies, peptides, enzymes, proteins, small molecules, cells, biological markers, DNA, peptides nucleic acid (PNA) (Achim, et al., 2009), locked nucleic acid (LNA), RNA, derivatives thereof, an indicator of an infectious disease, an indicator of a non-infectious disease, an indicator of a genetic disorder, an indicator of a newborn screening, an indicator of a prenatal screening, an indicator of a sexual transmitted disease, an indicator of a cancer, an indicator of the presence of pathogens, DNA, RNA, proteins, peptides, small molecules and any combination thereof, and cooligomers and any combinations thereof.

Examples of capture probes include, without limitation, antibodies, peptides, enzymes, proteins, small molecules, cells, biological markers, DNA, PNA, LNA, RNA, and derivatives, cooligomers and any combinations thereof.

Examples of detection probes include, without limitation, antibodies, peptides, enzymes, proteins, small molecules, cells, biological markers, DNA, PNA, LNA, RNA, and derivatives, cooligomers and any combinations thereof.

As used herein, “aggregation” of a group of subjects means an aggregation of one member subject of the group via covalent and/or non-covalent interaction(s).

As used herein, “combination” of a group of subjects means a mixture of any member subject of the group, or one subject or a mixture of subjects wherein each subject comprising one or more member subjects of the group.

As used herein, the term “conjugate” means a structure formed via covalent and/or non-covalent interaction(s) among two or more subjects, wherein the subjects can be the same or different, or the act to form the structure via covalent and/or non-covalent interaction(s) among two or more subjects; the term “conjugation” means formation of a structure via covalent and/or non-covalent interaction(s) among two or more subjects, wherein the subjects can be the same or different.

In certain embodiments, the nanoparticle has a structure with a high aspect ratio. For example, the nanoparticle may have a structure with greater than 1:3 aspect ratio. Examples of nanoparticles include, without limitation, nanofibers, nanowires, and nanosheets.

In certain embodiments, the nanoparticle comprises one or more molecules selected from the group consisting of conducting polymers and oligomers, non-conducting polymers and oligomers, and derivatives, copolymers, co-oligomers, and any combination thereof.

As used herein, the term “copolymer” means a polymer comprising more than one type of monomers. Examples of copolymers include, without limitation, block copolymers, random copolymers, alternating copolymers, periodic copolymers, statistical copolymers, linear copolymers and branched copolymers.

As used herein, the term “co-oligomer” means an oligomer comprising more than one type of monomers. Similar to copolymers, examples of co-oligomers include, without limitation, block co-oligomers, random co-oligomers, alternating co-oligomers, periodic co-oligomers, statistical co-oligomers, linear co-oligomers and branched co-oligomers.

Examples of conducting polymers and oligomers include, without limitation, polyaniline, polythiophene, polypyrrole, polyparaphenylenevinylene, oligoaniline, oligothiophene, oligopyrrole, oligoparaphenylenevinylene, and derivatives, copolymers, co-oligomers and any combinations thereof.

Examples of non-conducting polymers and oligomers include, without limitation, polystyrene, polyvinyl alcohol, polymethyl methacrylate, polysulfone, polyamides, polyacrylates, polystyrene sulfonic acid, oligostyrene, oligovinyl alcohol, oligomethyl methacrylate, oligosulfone, oligoamides, oligoacrylates, oligostyrene sulfonic acid, carbon nanotubes, carbon nanoscrolls, graphene, graphite, graphite oxide, silver nanowires, epoxy resins, organic dyes, and derivatives, copolymers, cooligomers and any combinations thereof.

In certain embodiments, the conjugation of the target molecules and the nanoparticle-based capture units results in aggregations of the nanoparticle-based detection units of multiple biosensors. Such aggregations are detectable by naked eye or by equipment (e.g. optical read-out equipment, microscope, UV-vis spectrophotometry, dynamic light scatterers, zeta potentiometers, viscometers, cyclic voltammogram, current-voltage read-out equipment, impedance read-out equipment, and potentiostat).

In certain embodiments, the nanoparticle-based capture unit of a biosensor comprises at least two capture probes. Each capture probe may conjugate to the same or different identifying sequences, and the identifying sequences may be on the same or different target molecules. Examples of such biosensors include, without limitation, biosensors comprising nanoparticles in a fibrullar or wire-like morphology with several capture probes.

### B) Preparation of Biosensors.

In certain embodiments, functionalizations of nanoparticle-based detection units with nanoparticle-based capture units are accomplished via covalent interactions. Examples of covalent interactions can be formed among, without limitations, carboxylic acid and amine/amino groups, aldehyde and amine/amino groups, carboxylic acid and alcohol groups, ketones groups, ethers, halides, carboxylic acid and thiol groups, thiol and thiol groups, biotin and streptavidin.

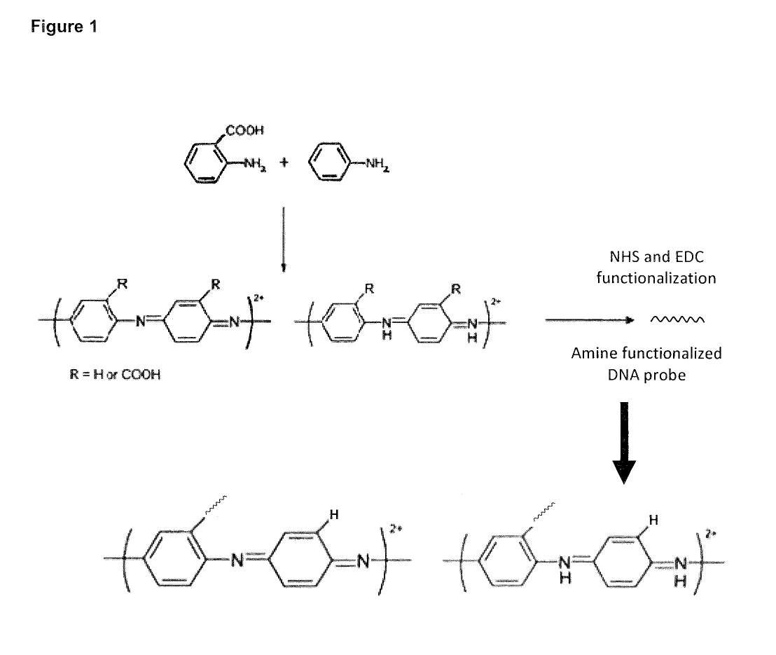

In certain embodiments, a functionalized polymer is prepared using functionalized monomer, non-functionalized monomers, functionalized oligomers, non-functionalized oligomers, and any combinations thereof. In certain embodiments, the total mole concentration of the functionalized monomer, functionalized oligomer or mixture thereof is about 0.001% to about 50%, about 0.01% to about 15%, about 0.01% to about 20%, or about 15% to about 50% of the total monomer mixture. In certain embodiments, conducting polymer nanofibers (e.g. polythiophenes, polypyrroles, polyanilines, and copolymers and derivatives thereof) containing desired functional groups are synthesized using additives (e.g. oligomers of the parent polymer or derivatives of the parent monomer) having the desired functional groups. For example, carboxylic acid functionalized polyaniline nanofibers are prepared according to FIG. 1. The carboxylic acid derivative of the aniline monomers can be replaced with monomers containing desired functional groups (e.g. anthranilic acid, benzoic acid, 3-amino-4-methylbenzoic acid, 2-aminothiophenol, aniline derivatives or non-aniline derivatives containing the desired functional groups) to prepare polyaniline nanofibers functionalized with desired functional groups.

Carboxylic acid functionalized polyaniline is further functionalized with an amine functionalized DNA probe using 1-ethyl-3-(3-dimethylaminopropy-1)-carbodiimide (EDC) and N-hydroxysuccinimide (NHS) or analogous thereof (FIG. 1). In certain embodiments, a different carbodiimide, triazolol, or other coupling agents is used.

In certain embodiments, when there are more than one functional groups in the same nanoparticle and the functional groups can react to each other, one of the reactive functional group is protected before the fuctionalization of the nanoparticle with probes (e.g. capture probes and/or detection probes), and then deprotected after the functionalization is complete. The protection and deprotection of a functional group is carried out using organic chemistry method.

In certain embodiments, a capture probe of a nanoparticle-based capture unit is a protein, which can be functionalized to a polymer nanoparticle via covalent attachment of the amine terminus of surface lysine groups or decoupled thiols groups. A thiolated monomer is incorporated into the polymer nanoparticles in a similar fashion as described in FIG. 1.

The degree and amount of functionalization on the polymer can be observed electrochemically, with TGA, IR, GC-MS, or other techniques depending on the polymer and type of functionalization.

### Bulk Functionalizations of Biomolecules to Conducting Polymer Nanofibers:

In certain embodiments, covalent attachment of DNA (or other capture probes) that contain at least one free amine or carboxylic acid group naturally, or through functionalization, is attached to conducting polymer nanofibers synthesized through a bulk scalable reaction (including, but not limited to: interfacial, rapidly mixed or initiated reactions) (Kaner, Huang, Weiller and Virji, 2005,Kaner, Li and Huang, 2007) via amine and carboxylic acid coupling chemistry. Nanofibers of polyaniline have a free amine terminus at the end of each polymer chain that is available for covalent attachment to a biological molecule with a carboxylic acid or aldehyde terminating group. Incorporation of a carboxylic acid group or thiol group occurs by copolymerizing an aniline monomer functionalized with one of these groups (such as anthranilic acid, benzoic acid, 3-amino-4-methylbenzoic acid, 2-aminothiophenol or other monomers containing an additional amine, thiol or carboxylic acid) with aniline in a rapidly mixed or interfacial reaction that may or may not contain an initiator molecule such as N-phenyl-p-phenylenediamine. An interfacial reaction is a reaction where the monomers and oligomers are dissolved in an organic solvent that is not miscible with water. The oxidant that is used to initiate chain growth is dissolved in acid.

In certain embodiments, in a rapidly mixed reaction the monomer or monomers, oxidant, and oligomers or other additives, if used, are all dissolved in aqueous solution, typically at a pH below three. The synthesis of the functionalized conducting polymer nanofibers is typically carried out with a 1:20 to 3:1 oxidant to monomer ratio, and at an approximately 1-20% concentration of monomer in an appropriate doping solution.

In certain embodiments, covalent functionalization of polyaniline nanofibers is carried out to the amine terminus of the polyaniline nanofibers or through carboxylic acids that are incorporated into the polyaniline using EDC and NHS coupling reagents. Carboxylic acids are incorporated by polymerization of anthranilic acid, 3-aminobenzoic acid, 3-amino-4-methylbenzoic acid, on their own or combined as copolymers with each other or with aniline.

In certain embodiments, shorter chains and nanofibers are achieved by sonication in a sonicating bath to provide more chain termini for functionalization. The fibers are shortened into oblong particles. In certain embodiments, shorter chains and nanofibers are achieved by increasing the ratio of oxidant to monomer in the polymerization.

In certain embodiments, carboxylic acid groups are incorporated into polyaniline to provide additional places on the polymer chain for the incorporation of capture probes. By using carboxylic acid derivative several different probes can be attached to a single nanofiber or polymer chain. One advantage of incorporating different levels of functionalization through the incorporation of carboxylic acids into the nanofiber, rather than only functionalizing the chain terminus, is that it is easier to modify the polymer nanofiber to succeed at different sensitivities and in various incarnations of a biosensor.

Conducting polymers such as polypyrrole, polythiophene, and polyaniline can also be functionalized by the use of monomer derivatives of the parent polymer to form a derivative polymer nanoparticle (containing only functionalized monomer) or a copolymer of several different monomers. In certain embodiments, the attachment of DNA to the carboxylated monomer can also be carried out prior to polymerization (Thierry Livache et al., 1994; T. Livache et al., 1995) or after polymerization.

In certain embodiments, polyaniline and polyaniline derivative nanofibers are prepared using aniline and 0-100% of an aniline derivative (e.g. anthranilic acid, 3-aminobenzoic acid, 3-amino-4-methylbenzoic acid, or mixtures thereof). Different reaction conditions including the use of different initiators, initiator concentrations and temperatures may be used to synthesize these aniline and aniline derivative nanofibers.

An initiator (e.g. N-phenyl-p-phenylenediamine, p-phenylenediamine, or a mixture thereof) is used at a concentration of 0.001-10% by mole monomer. In certain embodiments, more than 10% initiator is used. In certain embodiments, other initiators are used to create nanofibers of the conducting polymer nanofiber derivatives (Tran, Norris, D'Arcy, Tsang, Wang, Mattes and Kaner, 2008,Tran, Shin, Hong, D'Arcy, Kojima, Weiller and Kaner, 2007,Tran and Kaner, 2008). In certain embodiments, oxidants or additives such as bleach, carbon nanotubes, inorganic nanowires, are used to provide desired synthesis, structure, or dispersability of the nanoparticles. In certain embodiments, electrochemical growth and/or template and/or electrospinning are used to synthesis nanofibers.

Polyaniline is of interest due to the low cost of the monomer and synthesis as well as the stability of the polymer nanofibers in solution. However, for polyaniline containing carboxylic acid groups the amine terminus of the polyaniline chains may react with the carboxylic acids during the coupling reaction instead of the amines on the biological molecule being attached. This is prevented by the use of a protecting group such as di-tert-butyl dicarbonate in the presence of sodium bicarbonate to protect the terminal amines of the polyaniline chains. Polythiophene, polypyrrole, polyaniline, and their derivatives and copolymers in a nanostructured or water processable form can also be used for the biosensor applications described below.

In certain embodiments, a carboxylated polyaniline is prepared using additives such as anthranilic acid and 3-aminobenzoic acid in a rapidly mixed reaction. In certain embodiments, the mole concentration of carboxylated aniline derivative monomer is about 1% or less. In certain embodiments, the mole concentration of carboxylated aniline derivative monomer is about 1% to about 15%. In certain embodiments, the mole concentration of carboxylated aniline derivative monomer is about 15% or less.

In certain embodiments, the concentration of probe to be functionalized to a nanoparticle is from about 0.25 unit of probe per fiber to about 1000 units per fiber. The functionalization of the ssDNA is tested using cyclic voltammetry. The degree of functionalization of ssDNA is optimized for the sensitivity desired.

### II) A Biosensor System Comprising More Than One Type of Biosensors Targeting to the Same Target-Molecules.

Another aspect of the invention relates to a biosensor system comprising more than one type of biosensors, wherein each type of biosensors comprising a different type of capture probes for the same target-molecule.

### III) Signal-Enhanced Biosensor System Comprising a Biosensor and a Signal Enhancer.

Another aspect of the invention relates to a biosensor system comprising the biosensor and a signal enhancer which can conjugate with the biosensor-sample conjugate to enhance the detection signal. In certain embodiments, the signal enhancer comprises a conductive substrate for electrochemical detection, other nanofibers for a cross-linking or precipitation based reaction, or a porous or transparent substrate for optical detection.

In certain embodiments, the signal enhancer comprising a substrate functionalized with a capture probe that can conjugate with the target molecule.

In certain embodiments, the biosensor is further functionalized with a first detection probe. The signal enhancer comprises a substrate functionalized with a second detection probe. The second detection probe does not conjugate with any target molecules of the biosensor and conjugate with the first detection probe, and the conjugation of the first and the second detection probe provides an enhanced signal of the change of the nanoparticle-based detection unit.

### IV) Biosensor System Comprising a Biosensor and a Substrate-Based Capture Unit.

Another aspect of the invention relates to a biosensor system comprising one or more biosensors according to the present disclosure and a substrate-based capture unit, wherein the substrate-based capture unit comprises:
a substrate functionalized with one or more types of capture probes;
the capture probes can conjugate with the target molecules of the biosensors; and
the capture probes are deposited in a single spot on the substrate, or in an array on the substrate.

In certain embodiments, the substrate-based capture unit comprises the same type of capture probes. In certain embodiments, the substrate-based capture unit comprises at least two capture probes. Each capture probe may conjugate to the same or different identifying sequences, and the identifying sequences may be on the same or different target molecules.

In certain embodiments, the nanoparticle-based capture unit comprises the same type of capture probes. In certain embodiments, the nanoparticle-based capture unit comprises at least two capture probes. Each capture probe may conjugate to the same or different identifying sequences, and the identifying sequences may be on the same or different target molecules.

In certain embodiments, the biosensor system further comprises a signal enhancer, wherein:
the biosensor further comprises a first detection probe;
the signal enhancer comprises a substrate (e.g. a nanoparticle, a dipstick array, a porous substrate, a transparent substrate, a substrate coated with a transparent conductor substrate, a transparent conductor electrode substrate of an electrochemical monitoring equipment, a glass substrate, a silicon substrate, a mica substrate, a metal substrate, an Indium Tin Oxide (ITO) substrate, a cloth substrate, a paper substrate, a cotton substrate, a wood substrate, a cellulose substrate, polymeric film, a nitrocellulose substrate, a polyvinylidene fluoride substrate, a polystyrene sulfonic acid substrate, a poly-lysine substrate, a conductive substrate, a carbon nanotubes substrate, a graphene substrate, a silver nanowire substrate, a transparent conductor electrode substrate of potentiostat, and any combination thereof); and
   the conjugation of the first and the second detection probe provides an enhanced signal of the change of the nanoparticle-based detection unit.
      V) A Method of Detecting a Target Molecule from a Sample Using a Biosensor.

Another aspect of the invention relates to a method of detecting a target molecule from a sample comprising:
providing a biosensor or a biosensor system comprising a biosensor according to the present disclosure;
contacting the biosensor or a biosensor system with the sample to provide a biosensor-sample conjugate or a biosensor system-sample conjugate; and
detecting a detection signal of the biosensor-sample conjugate or the biosensor system-sample conjugate to determine the presence or concentration of the target molecule in the sample.

In certain embodiments, the detecting method further comprises:
detecting a first original detection signal of the biosensor and/or a second original detection signal of the sample; and
comparing the first detection signal of the biosensor-sample with the first original detection signal of the biosensor and/or the second original detection signal of the sample; and
determine the presence and/or the concentration of the target molecule in the sample.

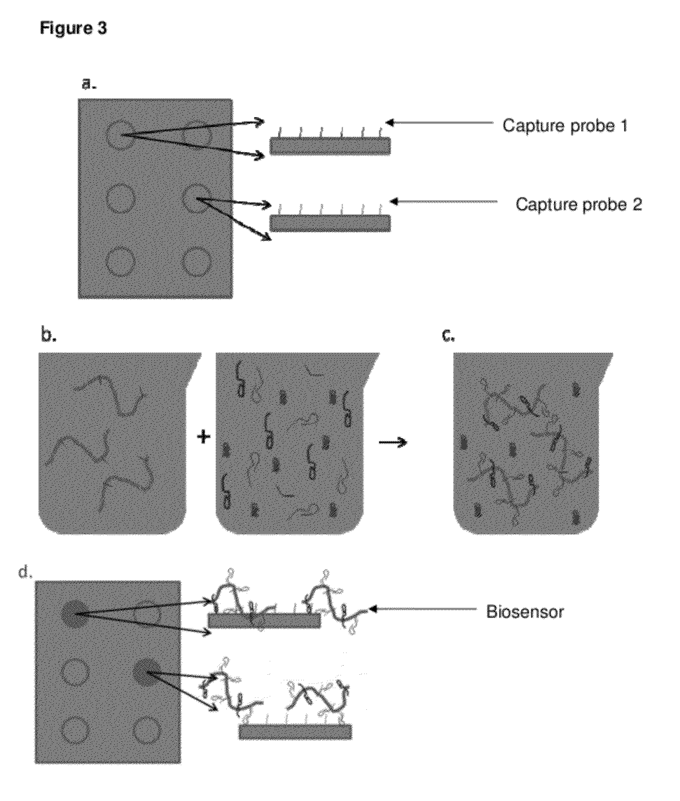

In certain embodiments, through optimizing the degree of functionalization (by altering the amount of carboxylic acid or amine groups available in the nanofiber for probe attachment) the presence of probe or capture molecule on the nanofibers and finding the appropriate concentration of nanofibers, the cross-linking reaction is tuned to occur at a specific concentration of target molecule, such as the point where there are at least 100,000 bacteria/mL of urine. This concentration of bacteria is typically used as the cut-off for diagnosing a UTI (FIG. 3). The nanofiber solutions before and after addition of salt show a stable dispersion and precipitation respectively. Though salt and other ions can cause a stable dispersion to settle over time, they are still semi-stable and can be dispersed briefly prior to settling out. When there is cross-linking between two conducting polymer nanofibers through the linkage of the target molecule to the capture molecules or probes, the nanofibers form a mass that does not resuspend in water with mild shaking.

In certain embodiments, the nanoparticle-based capture unit of the biosensor comprises at least two capture probes. Each capture probe may conjugate to the same or different identifying sequences, and the identifying sequences may be on the same or different target molecules. Examples of such biosensors include, without limitation, biosensors comprising nanoparticles in a fibrullar or wire-like morphology with several capture probes. The biosensor can be dispersed in a solution to form a semi-stable suspension in the absence of the target molecules. In the present target molecules, the biosensors conjugate with the target molecules to form biosensor-sample conjugate. The scattering profile of biosensor-sample conjugate is different comparing to uniform dispersions. Equipment such as UV-Vis spectrophotometer, dynamic light scatterers, zeta potentiometers, viscometers and microscopes may be used for improved accuracy and quantitative results in detecting the detection signal. In certain embodiments, the biosensor-sample conjugate is big enough to precipitate from the solution, which can be detected by naked eyes, or any equipment described supra for improved accuracy and quantitative results.

In certain embodiments, this method can be used in monitoring and detecting in an infectious disease, monitoring and detecting a non-infectious disease diagnosis, monitoring and detecting a genetic disorder diagnosis, monitoring and detecting a newborn screening, monitoring and detecting a prenatal screening, monitoring and detecting a sexual transmitted disease, diagnosis, monitoring and detecting a cancer, and monitoring and detecting conditions relating to pathogens, DNA, RNA, proteins, peptides, small molecules and any combination thereof in the samples. In certain embodiments, the method is also used in monitoring and detecting pathogens in water or food samples.

VI) A Method of Detecting a Target Molecule from a Sample Using a Biosensor System Comprising More Than One Type of Biosensors Targeting to the Same Target-Molecules.

Another aspect of the invention relates to a method of detecting a target molecule from a sample comprising:
providing a biosensor system comprising more than one type of biosensors, wherein each type of biosensors comprising a different type of capture probes for the same target-molecule;
contacting the biosensor system with the sample to provide a biosensor-sample conjugate comprising more than one nanoparticle-based detection units and more than one nanoparticle-based capture unit conjugated together via conjugation with the target-molecule at the same time; and
detecting a detection signal of the biosensor-sample conjugate to determine the presence or concentration of the target molecule in the sample.

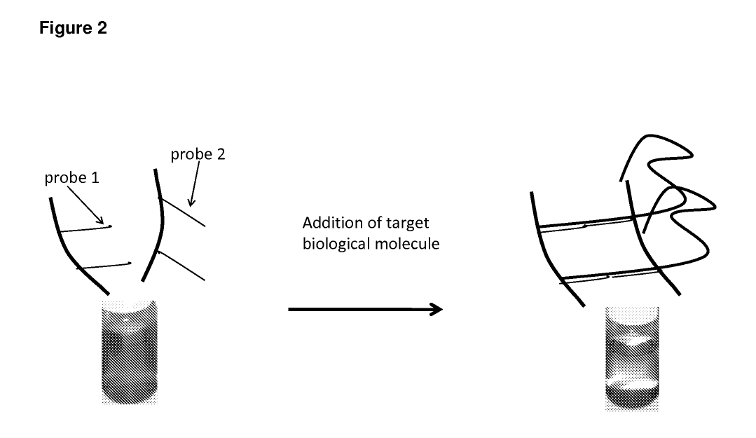

In certain embodiments, the biosensor system is dispersed in a solution to create a semi-stable dispersion that remains suspended for at least minutes. In the presence of the target molecules, capture probes of different biosensors conjugate to the same target molecule causes aggregation of the nanoparticle-based detection units (biosensor-sample conjugate). When the aggregation of the nanoparticle-based detection units is significant enough that the biosensor-sample conjugate can no longer remain suspended in the solution, the biosensor-sample conjugate will precipitate to provide a detection signal showing the presence of the target molecule in the sample (FIG. 2).

In certain embodiments, the nanoparticle of the biosensor comprises conducting polymer nanofibers (CPNFs) (FIG. 2).

In certain embodiments, the degree of probe functionalization can be controlled by altering the amount of functional group (e.g. carboxylic acid or amine groups) available in the nanofiber for probe functionalization. The degree of probe functionalization is optimized to provide detection signal at a specific concentration of target molecule in the sample (e.g. at least 100,000 bacteria/mL in urine, which is typically used as the cut-off for diagnosing a UTI) (FIG. 2). The nanofiber solutions before and after addition of salt show a stable dispersion and precipitation respectively. Though salt and other ions can cause a stable dispersion to settle over time, they are still semi-stable can be dispersed briefly prior to settling out. When there is conjugation between two conducting polymer nanofibers through the conjugation of the target molecule to the capture probes, the nanofibers form a mass that does not resuspend in water with mild shaking (FIG. 2).

In certain embodiments, the detection method is carried out using a single vial for inexpensive detection of the presence of a group of targets with a ubiquitous probe (for example a probe for gram negative bacteria or a universal pathogen probe).

In certain embodiments, an array of solutions is used with a well-plate or other small array for the detection of different targets. The array detection method can be used as a specific secondary test to determine the proper course of treatment for a patient found to have a UTI through identification of the type of organism and the antibiotic susceptibility of the organism.

In certain embodiments, this method can be used monitoring and detecting in an infectious disease, monitoring and detecting a non-infectious disease diagnosis, monitoring and detecting a genetic disorder diagnosis, monitoring and detecting a newborn screening, monitoring and detecting a prenatal screening, monitoring and detecting a sexual transmitted disease diagnosis, monitoring and detecting a cancer, and monitoring and detecting conditions relating to pathogens, DNA, RNA, proteins, peptides, small molecules and any combination thereof in the samples. In certain embodiments, the method is also used in monitoring and detecting pathogens in water or food samples.

In certain embodiments, because of the different scattering profile of nanoparticle-sample conjugate compared to uniform dispersions of the biosensors, a UV-Vis spectrophotometer can be used to detect the detection signal. A UV-Vis spectrophotometer can increase the accuracy and quantitative value of the test, particularly when low amounts of target molecule are attained. Other equipment such as dynamic light scatterers, zeta potentiometers, viscometers and microscopes may also be used to detect the detection signal with improved accuracy and quantitative results.

In another aspect of this embodiment, a non-colored polymer or nanoparticle can be used that suspends in water and is functionalize with a capture molecule. The material can either have dye incorporated into it, or would be visible with target induced cross-linking without addition of colorants. Similarly to the conducting polymers, nanoparticles in a fibrullar or wire-like morphology with several capture molecule sites are ideal to allow the best chance for interaction and binding of many particles.

VII) A Method of Detecting a Target Molecule from a Sample Using a Signal-Enhanced Biosensor System.

Another aspect of the invention relates to a method of detecting a target molecule from a sample comprising:
providing a signal-enhanced biosensor system comprising a signal enhancer and a biosensor;
contacting the signal-enhanced biosensor system with the sample to provide a signal-enhanced biosensor-sample conjugate; and
detecting a detection signal of the signal-enhanced biosensor-sample conjugate to determine the presence or concentration of the target molecule in the sample.

In certain embodiments, contacting the signal-enhanced biosensor system with the sample comprises:
contacting the biosensor with the sample to form biosensor-sample conjugate; and
contacting the biosensor-sample conjugate with the signal enhancer to form the signal-enhanced biosensor-sample conjugate

In certain embodiments, contacting the signal-enhanced biosensor system with the sample comprises:
contacting the signal enhancer with the biosensor to form signal enhanced biosensor conjugate; and
contacting the signal enhanced biosensor conjugate with the sample to form the signal-enhanced biosensor-sample conjugate
   VIII) A Method of Detecting a Target Molecule from a Sample Using a Biosensor System Comprising a Substrate-Based Capture Unit.

Another aspect of the invention relates to a method of detecting a target molecule from a sample comprising:
providing a biosensor system comprising a biosensor and a substrate-based capture unit;
contacting the biosensor system with the sample to provide a substrate-biosensor-sample conjugate; and
detecting a detection signal of the substrate-biosensor-sample conjugate to determine the presence or concentration of the target molecule in the sample.

In certain embodiments, contacting the biosensor system with the sample comprises contacting the biosensor with the sample to provide a biosensor-sample conjugate, and then contacting the biosensor-sample conjugate with the substrate-based capture unit to provide a substrate-biosensor-sample conjugate.

In certain embodiments, contacting the biosensor system with the sample comprises contacting the substrate-based capture unit with the sample to provide a substrate-sample conjugate, and then contacting the substrate-sample conjugate with the biosensor to provide a substrate-biosensor-sample conjugate.

In certain embodiments, the substrate-based capture unit comprises a dip-stick array type substrate (FIG. 3). Conducting polymers have high absorptivity and are easy to be detected at low concentration. Moreover, polyaniline nanofibers are easy to disperse in water and inexpensive to manufacture. The dipstick array substrate can be a glass, silicon, mica, metal, ITO, cloth, paper, cotton, wood, cellulose or other substrate or polymeric film (such as nitrocellulose, polyvinylidene fluoride, polystyrene sulfonic acid or poly-lysine)(Bailey, Kwong, Radu, Witte and Heath, 2007,Kwong, Bailey, Fan and Heath, 2008) that is free-standing or deposited on a substrate. The substrate material is modified with a capture probe via a self-assembled monolayer, thiol functionalization, silane functionalization, UV cross-linking, drop-casting, patterned deposition or through imbedding the capture probe into it (FIG. 3a). When a porous substrate is used where non-specific binding of the nanofiber might occur, the probe is spotted at different locations on the material and then immersed in polystyrene sulfonic acid, which prevents non-specific binding of the nanofibers to the substrate. Alternatively the substrate is immersed in polystyrene sulfonic acid or another polymer or functionalizing treatment prior to deposition of the probes. Substrates such as polyethylterrephthalate, nitrocellulose, polyvinylidene fluoride, and some other plastics are less likely to have non-specific attachment of conducting polymers and are thus ideal substrates. The capture probes on the biosensors conjugate with the target molecules to form biosensor-sample conjugate in solution (FIGS. 3b and 3c) if the target molecules are present in the sample. The biosensor-sample conjugate further conjugates with the substrate to form substrate-biosensor-sample conjugate (FIG. 3d). In certain embodiments, the target molecules can be described supra. In certain embodiments, the capture probes can be DNA and the target molecules can be 16S rRNA. Each spot on the dipstick is functionalized with a capture probe for a specific target molecule, and may have one or more spots in a specific spatial arrangement. The result is detection via the appearance of color attributed to the adhesion of the polymer to the spot for the corresponding target molecule.

In certain embodiments, the substrate is coated with a transparent conductor (e.g. a thin film of carbon nanotubes, graphene, silver nanowire, or ITO) an optical signal (e.g. color change) and an electrochemical signal on a potentiostat can be generated. The target molecule is conjugated with the capture probes on the transparent conductor coated substrate and those on the biosensors, an electrochemical signal from the redox activity of the conducting polymer will result. The presence of the target molecule would then cause an intense signal in a cyclic voltammogram or other current-voltage or impedance readings from the conducting polymer. The capture probes can be attached as described above in the optical read-out methods, or through attachment methods known in the field.

In certain embodiments, the biosensor system further comprises a signal enhancer capable of conjugating to the substrate-biosensor sample conjugates.

In certain embodiments, a method of detecting a target molecule from a sample comprising:
providing a biosensor system comprising a biosensor for the target molecule, a substrate-based capture unit and a signal enhancer;
contacting the biosensor system with the sample to provide a signal-enhanced substrate-biosensor-sample conjugate; and
detecting a detection signal of the signal-enhanced substrate-biosensor-sample conjugate to determine the presence or concentration of the target molecule in the sample.

In certain embodiments, contacting the biosensor system with the sample comprising:
contacting the biosensor with the target molecules and the substrate-based capture probe to provide a substrate-biosensor-sample conjugate; and
contacting the substrate-biosensor-sample conjugate with the signal enhancer to provide a signal-enhanced substrate-biosensor-sample conjugate.

In certain embodiments, contacting the biosensor system with the sample comprising:
contacting the signal enhancer with biosensor to provide a signal enhanced biosensor;
contacting the signal enhanced biosensor with the target molecules and the substrate-based capture probe to provide a signal enhanced substrate-biosensor-sample conjugate.

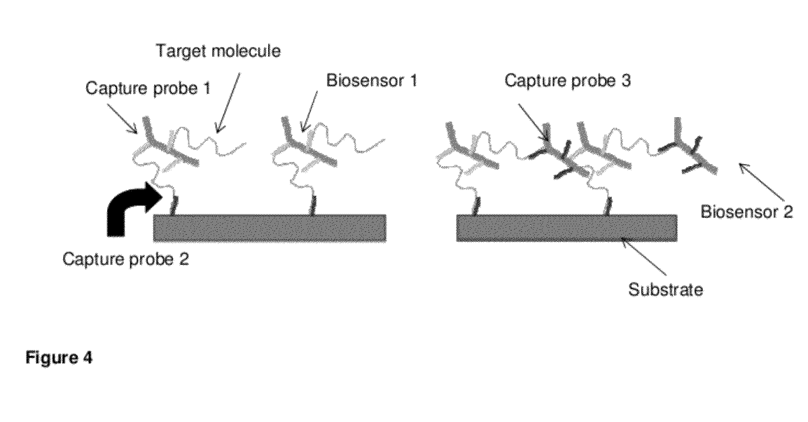

In certain embodiments, the signal enhancer comprising a substrate functionalized with a capture probe that can conjugate with the target molecule. In certain embodiments, the substrate is another nanoparticle (FIG. 4). Extra nanoparticle is conjugated into the signal enhanced substrate-biosensor sample conjugate, which causes an enhanced detection signal.

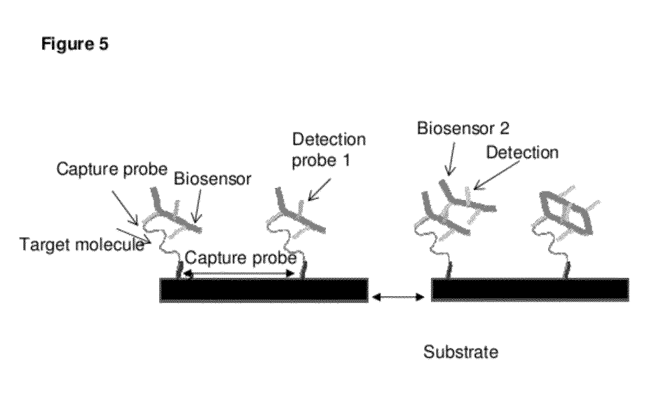

In certain embodiments, the biosensor is further functionalized with a first detection probe. The signal enhancer comprises a substrate functionalized with a second detection probe. The second detection probe does not conjugate with any target molecules of the biosensor and conjugate with the first detection probe, and the conjugation of the first and the second detection probe provides an enhanced signal of the change of the nanoparticle-based detection unit. In certain embodiments, the substrate is another nanoparticle (FIG. 5). Extra nanoparticle is conjugated into the signal enhanced substrate-biosensor sample conjugate, which causes an enhanced detection signal.

### IX) Electrochemical Readout of a Biosensor Array

Another aspect of the invention relates to electrochemical detection of target molecules using biosensors wherein the nanoparticles of the biosensors are conducting polymer nanostructures or nanofibers.

In certain embodiments, the detection method comprises template growth and/or electrochemical growth of the conducting polymer nanofibers, which then act as the active element in an electrochemical biosensor. The conducting polymer can be deposited on electrodes including gold, silver, aluminum, copper, platinum, iron, stainless steel etc. and electrochemical changes can be monitored based on hybridization to capture probes imbedded or covalently functionalized to the conducting polymer nanofibers. Deposition, through drop-casting, charge deposition, silk-screening, spray coating, spin-coating or other technique, could be used to deposit conducting polymer.

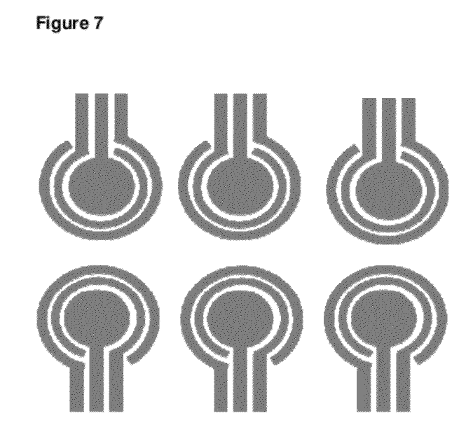

An electrode array with each electrode device having a central circular working electrode, and a surrounding counter and reference electrodes having a structure similar to FIG. 7 can be used. Because the probe that would capture the target molecule is covalently attached to the conducting polymer the electrodes do not have to be gold. Typical electrochemical devices involve probes with a thiol group and this is used to form a self-assembled monolayer on the gold surface. The probes on the surface then conjugate with the target molecule which further conjugates to a second probe containing a redox active material (e.g. peroxidase enzyme). Gold is both expensive and delicate, which can lead to device failure. Aluminum is an inexpensive and robust possibility, though any conducting material, as mentioned above, could potentially be used. Signal can be caused by uptake of the target and a second probe by the functionalized conducting polymer nanofibers to cause a redox reaction that is then amplified by the conducting polymer. The second probe can be functionalized with a redox active molecule such as a peroxidase, including horseradish peroxidase, a glucose oxidase, an acid group, a basic group or another group that changes either the oxidation state or doping level of the conducting polymer. Readout can be through a potentiostat to read the current-voltage curve changes or changes in the conductivity.

To employ conducting polymers in the electrochemical set-up set forth herein, one may include probes covalently bound to the substrate or probes doping or non-covalently attaching to the conducting polymer nanofibers.

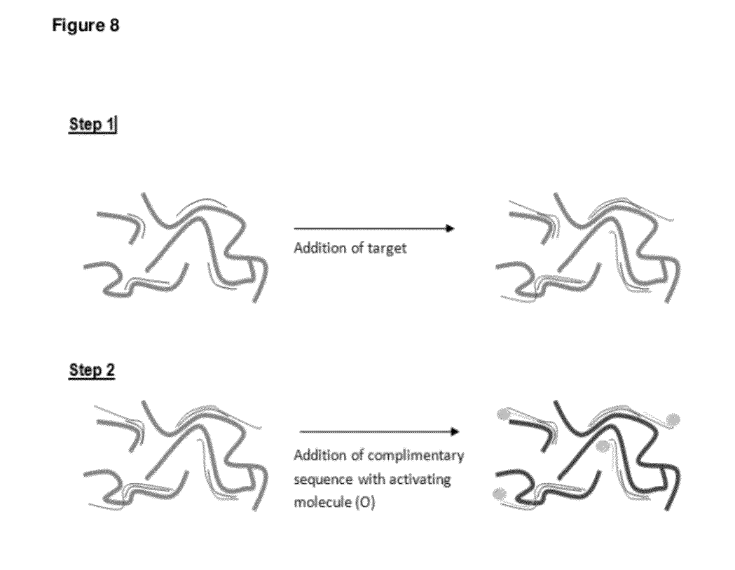

In certain embodiments, the redox activity of conducting polymers is used to amplify the signal found in DNA capture probe detection systems which use HRP and hydrogen peroxide (H₂O₂). The signal generated can be detected through either a potentiostat for an electrochemical signal, or a UV-Vis for color changes. HRP based conducting polymer biosensors can use an electrochemical-based signal, or a colorimetric based detection scheme if sensitivity is sufficient to decrease the cost of the system. One advantage of this system is the potential for changing the electrode to a less costly material than gold for array development. The steps of this detection method are illustrated in FIG. 8.

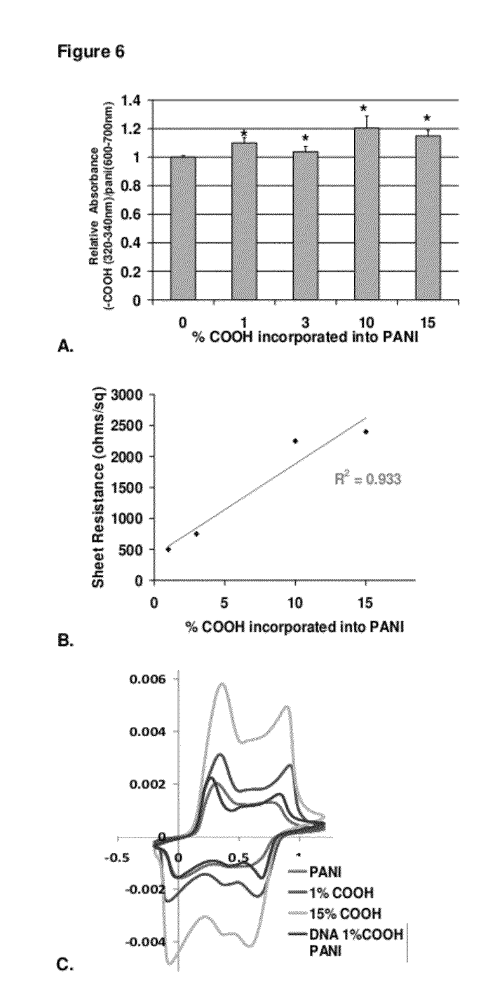

First, conducting polymer nanofibers can be functionalized covalently with capture probes, or by combination in solution and then deposited onto a substrate. In Step 1 of FIG. 6, the conducting polymer nanofibers are shown in green, the target nucleic acids are blue, and the probe tethered to polyaniline is red.

Second, the target molecule will bind to the capture probes. Electrical response is induced via oxidation of the conducting polymer nanofibers with the activation complex functionalized nucleic acid probe (orange) and an additional activating chemical (such as hydrogen peroxide) if needed.

Finally, HRP functionalized detector probes will be used to oxidize polyaniline in the presence of H₂O₂ to generate a signal from the oxidation of the conducting polymer. This is detectable with a potentiometer through constant voltage reading of current.

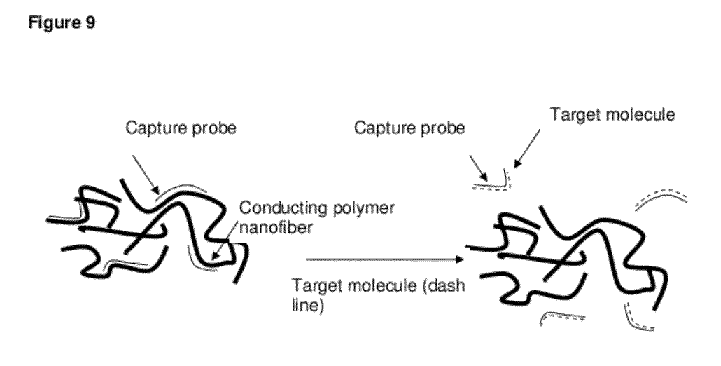

In certain embodiments, the biosensor comprises conducting polymer nanofibers doped with a capture probe as counterion. In certain embodiments, the biosensor is prepared by adding the capture probe during polymerization of the conducting polymer. In certain embodiments, the capture probe can be added after the nanobifers are prepared to dope the surface of the nanofibers. When a biosensor contact with a target molecule, the target molecule will conjugate with a capture probe and remove the capture probe from the nanofibers. Because the capture probe acts as a counterions in the nanofibers, removal of the counterions will demonstrate a change in current and capacitance of the conducting polymer in arrays and films, and the electrical response can be monitored (FIG. 9). In certain embodiments, the capture probe can be a desired DNA probe. In certain embodiments, the nanofibers are polypyrroles.

### EXAMPLES

The following examples are provided to better illustrate the claimed invention and are not to be interpreted in any way as limiting the scope of the invention. All specific compositions, materials, and methods described below, in whole or in part, fall within the scope of the invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the invention. It is the intention of the inventors that such variations are included within the scope of the invention.

### Example 1

### Synthesis of Carboxylated Polyaniline

COOH-functionalized polyaniline (COOH-PANI) was synthesized using the rapidly mixed method (Huang, 2006,Kaner, et al., 2007,Tran and Kaner, 2006) with addition of a carboxylic acid functionalized monomer (e.g. derivative of aniline, or other monomer having carboxylic acid group). An amount from 0.001% to 15% of 3-amino benzoic acid was dissolved with aniline into a 1 to 2 N acid solution. The acid used was sulfuric acid or hydrochloric acid, but can be any protonic or Lewis acid. The total monomer concentration is 0.05-0.5 Molar in the final reaction solution. An additive such as phenylene diamine, N-phenyl-p-phenylene diamine, aniline oligomer of up to 12 monomer units, a derivative of aniline, a derivative of an aniline oligomer, or a combination thereof were added at a concentration of 0-25%, preferably 1-5% by mole monomer by dissolving into the monomer acid solution. The reaction was then combined with some or no stirring with a solution of the oxidant dissolved in the same doping acid used for the monomer and additive. The amount of oxidant was typically four times the concentration of total monomer used. The oxidant solution was typically ammonium peroxydisulfate, but alternative oxidants such as bleach, iron chloride, gold chloride, potassium iodate, potassium biiodate, hydrogen peroxide or combinations of oxidants may also be used. The reactions were typically completed at a temperature between 14° C. and 30° C. (e.g. room temperature), and temperatures from −15° C. to 85° C. can be used.

### Example 2

### DNA Functionalized Polyaniline

COOH-PANI obtained in Example 1 was used to synthesize DNA functionalized polyaniline. The DNA probes were synthesized using standard methods and functionalized with an amino group at the 5′-terminus. The DNA probes were synthesized or purchased through a third party. A pre-determined concentration of carboxylated polyaniline and aminated DNA probes were used in the reaction in order to achieve the exact conjugation events for the optimal signal. COOH-PANI was coupled to the 5′amino-DNA probe using a standard EDC and NHS coupling reaction. Proof of functionalization is illustrated in FIG. 6.

### Example 3

### Biosensor with Conjugation Induced Precipitation for a Visual Readout

A) Sample comprising isolated RNA

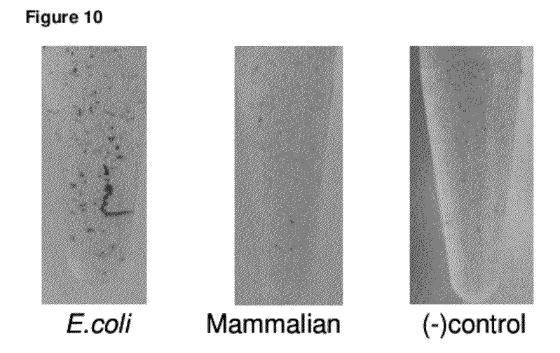

RNA was isolated from E. coli strain CFT073 using standard methods in the field. In addition, RNA from mammalian cells was used to show non-specific conjugation of biosensor with RNA. The total RNA was then added to a solution containing hybridization buffer and E. coli specific probe functionalized polyaniline. The samples containing RNA isolated from E. coli strain CFT073 formed a visible conjugate (precipitation) while the RNA from mammalian cells did not (FIG. 10).

B) Sample comprising lysed cells:

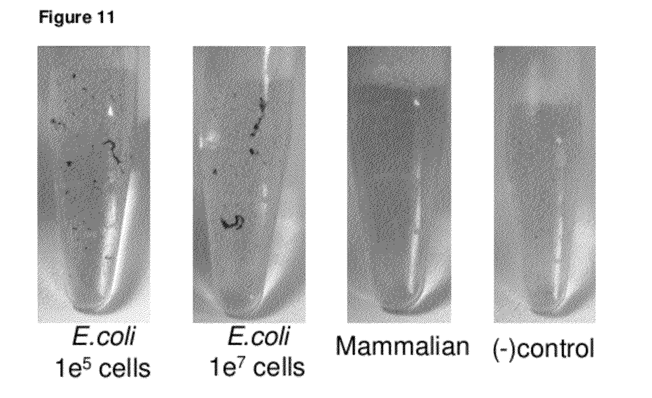

E. coli and mammalian cells were lysed using standard lysis techniques. The lysate was then added to a solution containing hybridization buffer and E. coli specific probe functionalized polyaniline. The samples containing lysates from E. coli formed a visible conjugation reaction while the lysates from mammalian cells did not (FIG. 11).

C) Urine samples:

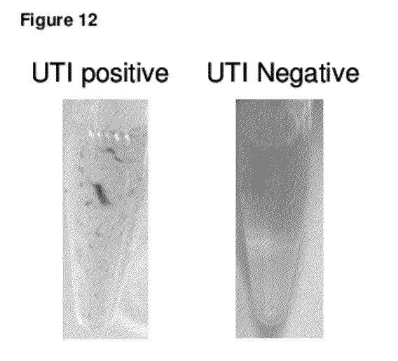

Standard lysis buffer was added to urine samples and then added to a solution containing hybridization buffer and E. coli specific probe functionalized polyaniline. The samples containing E. coli positive urine formed a visible conjugation reaction while UTI negative samples did not (FIG. 12).

### Example 4

### Biosensor where Target Molecules Are Target Cells

A) Target cells were Leukocytes:

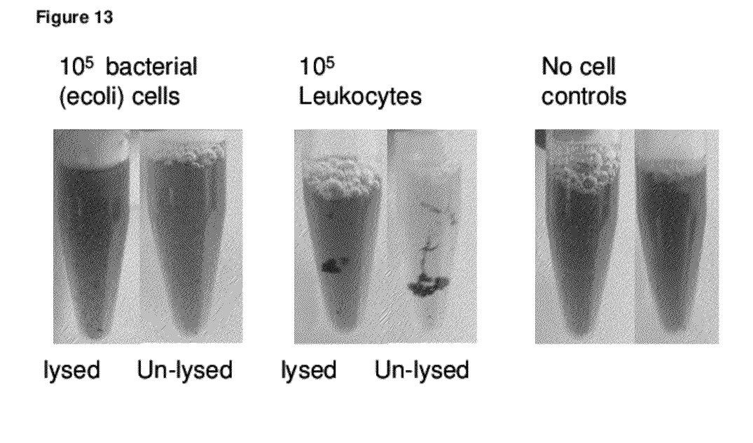

Leukocytes and E. coli cells were lysed using standard lysing procedures and polyaniline was then added to the lysates. The polyaniline nanofibers conjugated upon the presence of the lysate from Leukocytes and not E. coli (FIG. 13).

Polyaniline was added to Leukocytes and E. coli cells without any lysing procedures. The polyaniline nanofibers conjugated upon the presence of Leukocytes and not E. coli (FIG. 13).

### Example 5

### Detection of Target Molecule Using a Biosensor System Comprising a Biosensor (Probe Functionalized Polymer) and a Substrate-Based Capture Unit (Probe Functionalized Glass Slide)

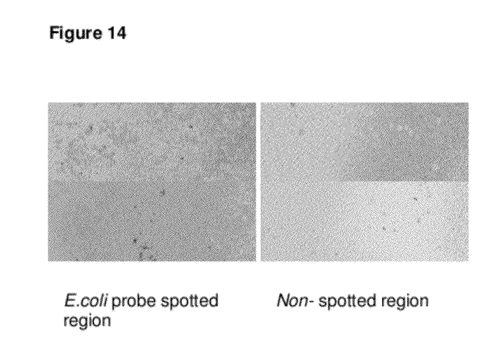

RNA was isolated from E. coli strain CFT073 using standard methods. The total RNA was then added to a solution containing hybridization buffer and E.coli specific probe functionalized polyaniline. The conjugated RNA/DNA polyaniline was then incubated with a glass slide spotted with specific E. coli probe, wherein the specific E. coli probe was proximal to the probe functionalized to the polyaniline. The RNA/DNA polyaniline hybridize to the specific spots on the glass slides that have been spotted with the E. coli probe (FIG. 14).

### REFERENCES

The following references are incorporated herein by reference in their entirety:
1 Achim, C., Shi, H., & Yeh, J. I. Biosensors and Related Methods. US Publication No. 20090061451 (2009).
2 Bailey, R. C., Kwong, G. A., Radu, C. G., Witte, O. N., & Heath, J. R., DNA-Encoded Antibody Libraries: A Unified Platform for Multiplexed Cell Sorting and Detection of Genes and Proteins. Journal of American Chemical Society 129 (7), 1959-1967 (2007).
3 Chang, H., Yuan, Y., Shi, N., & Guan, Y., Electrochemical DNA Biosensor Based on Conducting Polyaniline Nanotube Array. Analytical Chemistry 79 (13), 5111-5115 (2007).
4 Gao, Z. Nucleic Acid Biosensor with Photoelectrochemical Amplification. PCT Publication No. WO/2007004985 (2007).
5 Gao, Z., Xie, H., & Yu, Y. H. Method and Device for Detection of Nucleic Acids and/or Polypeptides. PCT Publication No. WO/2005106034 (2005).
6 Goel, A. Nano-PCR: Methods and devices for nucleic acid amplification and detection. US Publication No. US20060019274 (2006).
7 Haake, D. A. et al. Probes and Methods for Detection of Pathogens and Antibiotic Resistance. US Publication No. US20080199863 (2008).
8 Huang, J., Synthesis and applications of conducting polymer polyaniline nanofibers. Pure and Applied Chemistry 78 (1), 15-27 (2006).
9 Kaner, R. B., Huang, J., Weiller, B. H., & Virji, S. Synthetic Method for Conducting Polymer Nanofibers. US Publication No. US20050131139 (2005).
10 Kaner, R. B., Li, D., & Huang, J. Fabrication of polyaniline nanofiber dispersions and films. US Publication No. US20070194286 (2007).
11 Kwong, G., Bailey, R., Fan, R., & Heath, J.R. Methods and systems for detecting and/or sorting targets. PCT Publication No. WO/2008016680 (2008).
12 Labgold, M. R. et al. Device and Methods for Detecting and Quantifying One or More Target Agents. PCT Publication No. WO/2007092552 (2007).
13 Liao, J. C., Haake, D. A., Churchill, B. M., & Suchard, M. A. Rapid Species-specific Detection of Pathogens. PCT Publication No. WO/2006050275 (2006).
14 Liao, J. C. et al., Use of electrochemical DNA biosensors for rapid molecular identification of uropathogens in clinical urine specimens. J. Clin. Microbiol. 44 (2), 561-570 (2006).
15 Liao, J. C. et al., Development of an advanced electrochemical DNA biosensor for bacterial pathogen detection. J. Mol. Diagn. 9 (2), 158-168 (2007).
16 Livache, T. et al., Biosensing effects in functionalized electroconducting conjugated polymer layers: addressable DNA matrix for the detection of gene mutations. Synthetic Metals 71 (1-3), 2143-2146 (1995).
17 Livache, T. et al., Preparation of a DNA matrix via an electrochemically directed copolymerization of pyrrole and oligonucleotides bearing a pyrrole group. Nucleic Acids Research 22 (15), 2915-2921 (1994).
18 Mastali, M. et al., Optimal probe length and target location for electrochemical detection of selected uropathogens at ambient temperature. J. Clin. Microbiol. 46 (8), 2707-2716 (2008).
19 Musho, M. K., Noell, J. O., & Tse, P. H. S. Conductive sensors and their use in diagnostic assays. EP. Patent No. 467219 (1992).
20 Sun, C.-P. et al., Rapid, species-specific detection of uropathogen 16S rDNA and rRNA at ambient temperature by dot-blot hybridization and an electrochemical sensor array. Mol. Genet. Metab. 84 (1), 90-99 (2005).
21 Tran, H. D. & Kaner, R. B., A general synthetic route to nanofibers of polyaniline derivatives. Chemical Communications (37), 3915-3917 (2006).
22 Tran, H. D. et al., Substituted Polyaniline Nanofibers Produced via Rapid Initiated Polymerization. Macromolecules (Washington, DC, U. S.) 41 (20), 7405-7410 (2008).
23 Tran, H. D. et al., A template-free route to polypyrrole nanofibers. Macromolecular Rapid Communications 28 (24), 2289-2293 (2007).
24 Tran, H. H. D. & Kaner, R. B. Nanofibers from polyaniline derivatives and methods of synthesizing and using the same. US Publication No. US20080233400 (2008).
25 Wang, J., Jiang, M., Fortes, A., & Mukherjee, B., New label-free DNA recognition based on doping nucleic-acid probes within conducting polymer films. Analytica Chimica Acta 402 (1-2), 7-12 (1999).
26 Yang, T. et al., Synergistically improved sensitivity for the detection of specific DNA sequences using polyaniline nanofibers and multi-walled carbon nanotubes composites. Biosensors and Bioelectronics 24 (7), 2165-2170 (2009).
27 Zhu, N., Chang, Z., He, P., & Fang, Y., Electrochemically fabricated polyaniline nanowire-modified electrode for voltammetric detection of DNA hybridization. Electrochimica Acta 51 (18), 3758-3762 (2006).

1. A biosensor comprising a nanoparticle-based detection unit functionalized with a nanoparticle-based capture unit, wherein:
the nanoparticle-based detection unit comprises a nanoparticle;
the functionalization is through covalent or non-covalent interaction;
the nanoparticle-based capture unit comprises one or more capture probes, the conjugation of target molecules with the capture probes of the nanoparticle-based capture unit result in a change of the nanoparticle-based detection unit, which provides a nanoparticle-based detection signal; and
the change of the nanoparticle-based detection unit is selected from the group consisting of a morphological change, a color change, an optical change, an electrochemical change, and any combination thereof.

2. The biosensor according the claim 1, wherein the nanoparticle is selected from the group consisting of nanofiber, nanowire, and nanosheet.

3. The biosensor according to claim 2, wherein the nanoparticle has a structure with greater than 1:3 aspect ratio.

4. The biosensor according to claim 1, wherein the nanoparticle comprises one or more molecules selected from the group consisting of conducting polymers and oligomers, non-conducting polymers and oligomers, and derivatives, copolymers, co-oligomers, and any combinations thereof.

5. The biosensor according to claim 4, wherein the conducting polymers and oligomers are selected from the group consisting of polyaniline, polythiophene, polypyrrole, polyparaphenylenevinylene, oligoaniline, oligothiophene, oligopyrrole, oligoparaphenylenevinylene, and derivatives, copolymers, co-oligomers, and any combinations thereof.

6. The biosensor according to claim 4, wherein the non-conducting polymers and oligomers are selected from the group consisting of polystyrene, polyvinyl alcohol, polymethyl methacrylate, polysulfone, polyamides, polyacrylates, polystyrene sulfonic acid, oligostyrene, oligovinyl alcohol, oligomethyl methacrylate, oligosulfone, oligoamides, oligoacrylates, oligostyrene sulfonic acid, carbon nanotubes, carbon nanoscrolls, graphene, graphite, graphite oxide, silver nanowires, epoxy resins, organic dyes, and derivatives, copolymers, co-oligomers, and any combinations thereof.

7. The biosensor according to claim 1, wherein:
the capture probes is selected from the group consisting of antibodies, peptides, enzymes, proteins, small molecules, cells, biological markers, DNA, PNA, LNA, RNA, and derivatives, cooligomers and any combinations thereof; and
the target molecule is selected from the group consisting of antibodies, peptides, enzymes, proteins, small molecules, cells, biological markers, DNA, PNA, LNA, RNA, derivatives thereof, an indicator of an infectious disease, an indicator of a non-infectious disease, an indicator of a genetic disorder, an indicator of a newborn screening, an indicator of a prenatal screening, an indicator of a sexual transmitted disease, an indicator of a cancer, an indicator of the presence of pathogens, DNA, RNA, proteins, peptides, small molecules and any combination thereof, and cooligomers and any combinations thereof.

8. The biosensor according to claim 1, wherein the conjugation of the target molecules and the nanoparticle-based capture units results in aggregation of the nanoparticle-based detection units of multiple biosensors.

9. The biosensor according to claim 8, wherein the aggregation of the nanoparticle-based detection units of multiple biosensors is detectable by naked eye or equipment selected from the group consisting of optical read-out equipment, microscope, UV-vis spectrophotometry, dynamic light scatterers, zeta potentiometers, viscometers, cyclic voltammogram, current-voltage read-out equipment, impedance read-out equipment, and potentiostat.

10. The biosensor according to claim 1, wherein the nanoparticle-based capture unit comprises at least two capture probes, and each capture probe can conjugate to different identifying sequences on the same target molecule.

11. A biosensor system comprising more than one type of biosensor according to claim 1, wherein each type of the biosensors comprises a different type of capture probes that can conjugate to the same target molecule.

12. A signal-enhanced biosensor system comprising a biosensor according to claim 1 and a signal enhancer, wherein:
the biosensor further functionalized with a first detection probe;
the signal enhancer comprises a substrate functionalized with a second detection probe which can conjugate with the first detection probe; and
the conjugation of the first and the second detection probe provides an enhanced signal of the change of the nanoparticle-based detection unit.

13. The signal-enhanced biosensor system according to claim 12, wherein the substrate is selected from the group consisting of a nanoparticle, a dipstick array, a porous substrate, a transparent substrate, a substrate coated with a transparent conductor substrate, a transparent conductor electrode substrate of an electrochemical monitoring equipment, a glass substrate, a silicon substrate, a mica substrate, a metal substrate, an ITO substrate, a cloth substrate, a paper substrate, a cotton substrate, a wood substrate, a cellulose substrate, polymeric film, a nitrocellulose substrate, a polyvinylidene fluoride substrate, a polystyrene sulfonic acid substrate, a poly-lysine substrate, a conductive substrate, a carbon nanotubes substrate, a graphene substrate, a silver nanowire substrate, a transparent conductor electrode substrate of potentiostat, and any combination thereof.

14. A biosensor system comprising one or more biosensors according to claim 1 and a substrate-based capture unit, wherein the substrate-based capture unit comprises:
a substrate functionalized with one or more types of capture probes;
the capture probes can conjugate with the target molecules of the biosensors; and
the capture probes are deposited in a single spot on the substrate, or in an array on the substrate.

15. The biosensor system according to claim 14, wherein:
the substrate-based capture unit comprises one or more types of capture probes; and
the nanoparticle-based capture unit comprises one or more types of capture probes.

16. The biosensor system according to claim 15, wherein the different types of capture probes of the nanoparticle-based capture units target to the same or different target molecules.

17. The biosensor system according to claim 14, further comprising a signal enhancer wherein
the biosensor further comprises a first detection probe;
the signal enhancer comprises a substrate selected from the group consisting of a nanoparticle, a dipstick array, a porous substrate, a transparent substrate, a substrate coated with a transparent conductor substrate, a transparent conductor electrode substrate of an electrochemical monitoring equipment, a glass substrate, a silicon substrate, a mica substrate, a metal substrate, an ITO substrate, a cloth substrate, a paper substrate, a cotton substrate, a wood substrate, a cellulose substrate, polymeric film, a nitrocellulose substrate, a polyvinylidene fluoride substrate, a polystyrene sulfonic acid substrate, a poly-lysine substrate, a conductive substrate, a carbon nanotubes substrate, a graphene substrate, a silver nanowire substrate, a transparent conductor electrode substrate of potentiostat, and any combination thereof; and
the conjugation of the first and the second detection probe provides an enhanced signal of the change of the nanoparticle-based detection unit.

18. A method of detecting a target molecule from a sample comprising:
providing a biosensor according to claim 1;
contacting the biosensor and the sample to provide a first biosensor-sample conjugate; and
detecting a first detection signal of the first biosensor-sample conjugate to determine the presence or concentration of the target molecule in the sample.

19. The method according to claim 18, wherein:
the target molecules are indicators of conditions selected from the group consisting of infectious diseases, non-infectious diseases, pathogens, genetic disorders, newborn screening, prenatal screening, sexual transmitted diseases, cancer, DNA, RNA, proteins, peptides, and small molecules; and
the sample is selected from the group consisting of water, food, blood, urine, cerebrospinal fluid, mucous, biopsies, biological specimens and bodily fluids.

20. A method of detecting a target molecule from a sample comprising:
providing a signal-enhanced biosensor system according to claim 11;
contacting the signal-enhanced biosensor system with the sample to provide a signal-enhanced biosensor-sample conjugate; and
detecting a detection signal of the signal-enhanced biosensor-sample conjugate to determine the presence or concentration of the target molecule in the sample.

21. A method of detecting a target molecule from a sample comprising:
providing a biosensor system according to claim 14;
contacting the biosensor system with the sample to provide a substrate-biosensor-sample conjugate; and
detecting a detection signal of the substrate-biosensor-sample conjugate to determine the presence or concentration of the target molecule in the sample.
